(19) United States
(12) Patent Application Publication
(10) Pub. No.: US 2003/0072798 A1
(43) Pub. Date: Apr. 17, 2003

(54) Solid self-emulsifying dosage form for improved delivery of poorly soluble hydrophobic compounds and the process for preparation thereof

(75) Inventor: Joseph Schwarz, Richmond Hill (CA)

Correspondence Address: OGILVY RENAULT, 1981 MCGILL COLLEGE AVENUE, SUITE 1600, MONTREAL, QC, H3A2Y3, CA

(73) Assignee: AlphaRx Inc., Richmond Hill (CA)

(21) Appl. No.: 10/252,158

(22) Filed: Sep. 23, 2002

Related U.S. Application Data
(63) Continuation-in-part of application No. 09/482,109, filed on Jan. 13, 2000, now abandoned.

(51) Int. Cl. A61K 9/64; A61K 9/20

(52) U.S. Cl. 424/456; 424/465

(57) ABSTRACT

A delivery method and product for enhancing the bioavailability of an active ingredient by prolonged relatively constant release. The method involves mixing with subsequent granulation and compression of a mixture to result in a solid core tablet. The composition includes a biologically active material matrixed or otherwise contained within a hydrophobic phase with the latter absorbed onto a sorbent. The sorbent and hydrophobic phase are in a ratio of between 1:10 and 10:1. The mixture further includes a pharmaceutically acceptable surfactant. The composition, once tableted into a solid core provides spontaneous release of the biologically active material over a predetermined time frame for substantially constant bioavailability.

### CROSS-REFERENCE TO RELATED APPLICATIONS

This is a continuation-in-part of U.S. Ser. No. 09/482,109, filed Jan. 3, 2002, which is in turn a continuation application of Ser. No. 09/482,109 filed Jan. 13, 2002.

### FIELD OF THE INVENTION

The present invention relates to a dry solid oral dosage form of biologically active substance in an oil phase of an oil in water emulsion and more particularly, the present invention provides regulated release of the active substance achievable on contact with water of body fluids.

### BACKGROUND OF THE INVENTION

Low bioavailability of hydrophobic drugs with extremely low water solubility can be a serious problem. Different approaches have been taken to achieve a desired level of drug solubility and dissolution rate. These approaches have been based on preparations with increased surface area (micronised powders), molecular inclusion complexes (cyclodextrines and derivatives), co-precipitates with water-soluble polymers (PEG, polozamers, PVP, HPMC) and non-electrolytes (urea, mannitol, sugars etc.), micellar solutions in surfactant systems (Cremophor™, Tween™, Gellucires™), multilayer vesicles (liposomes and niosomes). Dispersed colloidal vehicles, such as oil-in-water, water-in-oil and multiple (O/W/O or W/O/W) emulsions, microemulsions and self-emulsifying compositions also have been used to improve bioavailability of poorly soluble molecules.

None of these approaches has provided the efficiency for selected cases for bioavailability improvement of immediate drug release formulations. Moreover, a significant increase in bioavailability for such low soluble drugs as nifedipine can lead to dangerous side effects due to “dose dumping” . when the water miscible vehicle (PEG-400) has been used.

Self-emulsifying drug delivery systems usually comprise a mixture of the liquid or semi-solid lipid phase (usually fatty acid glycerides or esters) with a surfactant (e.g., oxyethylated glycerides or oxyethylated fatty acids), and an additional cosurfactant or cosolvent (e.g., lecithin, monoglycerides, aliphatic alcohols, PEO-PPG copolymers). A hydrophobic drug can be efficiently dissolved in the mixture. After the addition of water, the mixture rapidly converts into an oil-in-water emulsion with the drug remaining in the oil droplets. Absorption of the drug in gastro-intestinal system from the emulsion is increased.

Microemulsion systems are to some extent similar to a self-emulsifying system and often are composed of analogous components (oil, surfactant, short or medium chain alcohol as cosurfactant, and water) with the difference being in the ratio of the components. When diluted with water, an oil-in-water or water-in-oil emulsion may be produced, accordingly to composition and water aount. Drug entrapment and distribution in the stomach and intestine is also good.

All of the delivery systems discussed are liquid preparations and as such, the formulation must be administered as a fluid mixture or as a soft gelatine capsule (SGC).

Although useful, liquid and SGC present complications in turn of taste masking, compatibility with SGC walls, dosage from stability and manufacturing restraints.

Tableted forms of abovementioned delivery systems are limited to matrix type tablets, which do not provide any significant improvement of bioavailablity.

In the prior art, namely U.S. Pat. No. 5,897,876, issued Apr. 27, 1999 to Rudnic et al., there is disclosed an emulsified drug delivery system which specifically relates to a water-in-oil emulsion which contains a discontinuous water phase in an amount of between 5.1 and 9.9%. The examples are all directed to liquid compositions. Since the compositions are all liquid there is inherently a hydrophilic phase. In terms of tablet or solid discussion, Rudnic et al. only teach that the water emulsion could be absorbed on tablet excipients. This is significantly different from providing a tablet which is a homogenous composition emulsifiable in the presence of body fluid. In this respect, the Rudnic et al. disclosure is simply directed to a coating on a preformed tablet. The only area where the composition would be marginally homogeneous would be the exterior layer of the preformed tablet.

In terms of other advancements in this field, U.S. Pat. No. 6,174,547, issued Jan. 16, 2001, to Dong et al. teaches a liquid composition comprising a hydrophilic phase retained in a osmotic hydrogel matrix. This reference is primarily focused on a two phase emulsion. This is a significant departure from an emulsifiable composition. The composition set forth in the reference is not emulsifiable, since the composition is already emulsified in its liquid form. In this manner, Dong et al. do not address the complications associated with providing a homogeneous distribution within a tablet, which composition can be emulsified under certain conditions.

In Friedman et al., U.S. Pat. No. 6,004,566, issued December 1999, there is disclosed a topical emulsion cream. The emulsion is designed for transdermal delivery. Friedman et al. is only relevant to emulsions; there is nothing in the reference which would provide one skilled in the art with instruction to form a tableted emulsifiable composition.

There are numerous further references directed to sustained release formulations, water dispersible vitamin E compositions, etc. These reference include the following: U.S. Pat. Nos. 5,965,160; 5,858,401; 4,369,172; 4,259,314; 5,603,951; 5,583,105; 5,433,951; and 5,234,695.

It would be desirable to have a dry tablet formulation with a significant increase in bioavailability. The present invention addresses this requirement.

### SUMMARY OF THE INVENTION

The one object of the present invention is to provide an improved solid tablet and method of forming this tablet to enhance the bioavailability of an active ingredient over a prolonged period of time.

It has been found that the composition based on proper mixture of hydrophobic active compound with oil phase and surfactant (or combination of surfactants) and physiologically acceptable excipients, explicitly specific sorbents, can be successfully fabricated as dry solid tablet. Such tablets can be easily manufactured using standard equipment—mixers, granulators, tablet presses. Being placed into the water-containing media the abovementioned tablet generates “in situ” formulation of oil-in-water emulsion with active components dissolved in the oil droplets of the formed emulsion.

One object of one embodiment of the present invention is to provide claim 1

Advantageously, the pharmacokinetics of a biologically active compound can be influenced by the formulation of the tablet. It has been found that by providing a homogeneous dispersion of known compounds which are subsequently granulated and compressed into a hard solid body tablet that prolonged release is achievable.

As generally discussed herein previously, where prolonged release is attainable, blood “dumping” or rapid delivery of the biologically active material into the blood plasma can be avoided.

Where prolonged release is achievable, it follows that the bioavailability will demonstrate concomitant efficacy. It will be evident where this union of desirable results is realized, the patient to which the drug is administered does not have to be continuously interrupted for administration of, for example, a drug in order for the drug content in blood plasma levels to be sustained. This inherently leads to fewer doses over a predetermined time frame without. Where the bioavailability of the drug can be sustained in a substantially constant concentration the efficacy is not perceived to fluctuate by the patient. This provides the patient with comfort and regular metabolism of the drug over a time period.

In the tests conducted for the present invention, it was determined that the self-emulsifying tablet consistently maintained a higher active ingredient concentration in blood plasma for the same time frame for a non emulsifiable tablet.

Dissolution rate can be regulated by known to skilled person ways, e.g. using of water swellable eroding polymers or by other techniques, and sustained release of hydrophobic drug can be effectively suspended for desired time interval. Immediate release tablets also can be prepared by using of appropriate addition of disintegrants.

A further object of one embodiment of the present invention is to provide claim 2

With respect to the composition, successful results have been obtained with the composition when the same is a homogeneous mixture of the compounds in the composition. In one embodiment, the ingredients may be granulated and subsequently compressed into a tablet having a substantially uniform solid cross-section. This effectively provides uniformity which is important for effecting the bioavailability of the composition and particularly, the biologically active compound.

In the prior art, this was not recognized; the prior art taught the formation of a tablet, however, the emulsion composition has been only deposited as a coating on the tablet. At best, such an arrangement provides for localized homogeneity of the emulsion in a thin layer. This is vastly different from a composition which is entirely emulsifiable. When the tablet is entirely emulsifiable, prolonged release is achievable with relatively constant bioavailability. In the prior art formulations, localized homogeneity effectively provides active material “dumping” upon immediate dissolution with a rapid tapering of bioavailability.

The formulation of an emulsifiable composition is not without its complications. One of the more difficult challenges in preparation relates to the compression of the granulation. As is known, compression of materials into a tablet form requires enormous forces. In the instant composition, it was observed that the hydrophobic phase was not disrupted nor where the submicron particles containing the active ingredient when exposed to the compression for tableting.

Having thus described the invention, reference will now be made to the accompanying drawings illustrating preferred embodiments.

### BRIEF DESCRIPTION OF THE DRAWINGS

FIG. 1 is a graphical representation of the dissolution rate of coenzyme Q-10 self-emulsifying controlled release tablet;

FIG. 2 is a graphical representation of the dissolution rate similar to FIG. 1 using a 50 mg tablet;

FIG. 3 is a graphical representation of the dissolution rate of coenzyme Q-10 for different pH;

FIG. 4 is a graphical representation of the dissolution data for a variety of capsules;

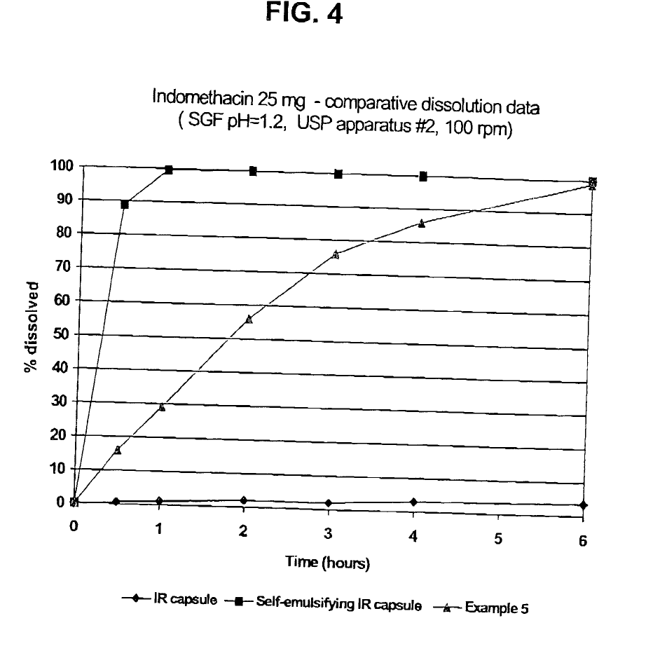

FIG. 5 is a graphical representation of comparative pharmacokinetics for coenzyme Q-10 tablets; and

FIG. 6 is a graphical representation of the particle size distribution for a self emulsifying tablet.

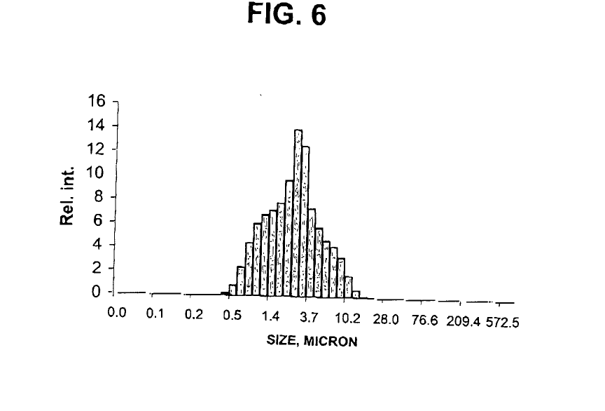

### DETAILED DESCRIPTION OF THE PREFERRED EMBODIMENTS

The lipid phase can be prepared from any physiologically acceptable oily or fatty component(s). It is desirable that the lipid phase is liquid or semisolid at body temperature to form an oil-in-water emulsion. As example, the lipid phase may comprise: triglycerides (food grade oils—live, corn, canola, soy; palm oil, cocoa oil, fractionated palm oil, medium chain triglycerides (MCT, capric/caprylic glycerides, etc.; animal fats, fish oil, tallow oil, modified glycerices—acetylated monoglycerides, mono- and digylcerides; lipid soluble vitamins—alpha-, beta- and gamma-tocopherol and correspondent tocopherol esters (vitamin E), tocotrienols and related compounds, retinol and retinol esters (vitamin A), etc.; aliphatic and aromatic esters: tributylcitrate, diethyladipate, dibutylphtalate, etc.

Miscellaneous lipid substances include Squalan, squalen, mineral oil, liquid silicon polymers, synthetic and natural waxes with a suitable melting point.

To form a tablet with suitable physico-chemical properties, an appropriate sorbent for the lipid phase must be used. The sorbent function is to hold the lipid phase during the granulation process to provide free flowing granulation, and prevent the lipid phase from leaking during the tableting process. The sorbent should be physiologically inert, safe and suitable for granulation and tabletting processes. The sorbent should possess high surface area/porosity, high mechanical strength and be relatively inert to prevent chemical interaction with formulation components. As example, the following compounds are typically suitable:

Silicon dioxide—colloidal (dried silicagel—Syloid™ 244, GRACE; Sipernats®, DEGUSSA) or fumed (prepared by hydrolysis of silicone alides -Cab-O-Sil® M5, CABOT, or Aerosil® 200/300, DEGUSSA), inorganic sorbents such as synthetic Magnesium Aluminum Silicate (Neusilin®, FUJI), di- and tribasic calcium phosphates, calcium carbonate, calcium silicate, zeolites, talcite, kaolin, benthonite, etc., cross-linked polymers with high surface area, such as cross-linked povidone (Povidone® XL, BASF) may also be used.

Biocompatible surfactants may selected from polyethoxylated derivatives of tocopherol acid succinate (TPGS™, East man-Kodak), glycerides (Gellucire™, Gatefosse, Tagat™, Henkel; etc), polyol esters (Sorbitan esters, Tween™), sucrose stearates (Sucrose ester™, Gattefosse), PEG derivatives of long chain acids (PEG stearate, Lipo-PEG™, Mirj® 52) or block-copolymers (Poloxamer™, Pluronic™) with suitable HLB value.

In respect of suitable excipients, sorbents, tablet forming materials, glidants, lubricants, hydration regulators can be selected according to desired tablet properties and loading level. Since many of the proposed components are liquid or semisolid materials at room temperature, preparation of the tablets becomes a challenging task. Highly absorptive compounds facilitate for preparation of free flowing powders, however, most of the absorbed material is squeezed out of the matrix during tablet compression (applied force is typically 1-10 tons per tablet), thus compromising the properties of the tablet.

The instant invention describes the preparation of the tablets with high of lipid and surfactant content. The tablet possesses acceptable physical characteristics such as hardness, friability, dissolution behavior and can be manufactured using standard equipment such as granulators, ovens, dryers, mixers, tablet presses. Upon contact with water or body fluid the tablet releases “in-situ”, forming an oil-in-water emulsion comprising an active component dissolved in an oil phase.

Such properties facilitate high bioavailability for hydrophobic substances, included into the tablet.

Polymers for release rate control work as main dissolution rate regulators. After contact with water they form a hydrated gel in parallel with emulsification process. Release of the formed emulsion follows the gel dissolution and partial diffusion of the tiny lipid droplets from gelled matrix to surrounded media. Preferred gel forming polymers are water swellable or water soluble cellulose derivatives, for example, Hydroxypropylmethylcellulose (Methocel™, types A, E, K, F, Dow Chemical), Hydroxyethylcellulose (Natrosol™, Hercules), Hydroxypropylcellulose (Klucel™, Aqualon), Carboxymethylcellulose (cellulose gum). Another types of synthetic polymers include polyacrylic acid (Carbopol™, BFGoodrich), Polyethylene oxide (Polyox™, Union Carbide), Polyvinylpyrrolidone (Kollidon™, PVP and PVP-VA, BASF), natural gums and polysaccharides—Xantan gum (Keltrol™, Kelco), carrageenan, locust bean gum, acacia gum, chitosan, alginic acid, hyaluronic acid, pectin, etc.

Having thus generally described the invention, reference will now be made to the examples.

### EXAMPLE 1

Coenzyme Q-10 Self-Emulsifying Controlled Release Tablet 30 mg strength, dissolution time>6 hours.

As a first example of the first formulation, the slowly dissolving composition contains Coenzyme Q-10 (Ubiquinone) in amount of 30 mg per tablet. The oil phase comprises of alpha-tocopherol acetate (vitamin E acetate), PEG-40 stearate (Lipo-PEG 39S) used as the surfactant with optimal HLB value for effective emulsification of the oil phase. A weight ratio of 1:1 between Q-10 and the oil phase was used. In respect of the surfactant to oil phase, the w/w ratio used was 1.6 to 1.

The composition of the 30 mg Q-10 self-emulsifying extended release tablet is displayed in table 1.

TABLE 1
Pharmaceutical Solid Self-Emulsifying Composition for
Sustained Delivery of Coenzyme Q-10 (30 mg tablet)

| | Per tablet, | |
|---|---|---|
| INGREDIENTS | mg | % |
| Coenzyme Q-10 | 30 | 6.41% |
| Tocopherol acetate | 30 | 6.41% |
| PEG-40 stearate | 50 | 10.68% |
| Dibasic calcium phosphate | 15 | 3.21% |
| Colloidal silicon dioxide (Cab-O- | 45 | 9.62% |
| Sil) | | |
| Lactose (spray dried) | 110 | 23.50% |
| Methocel E-15 | 24 | 5.13% |
| Methocel K4M | 48 | 10.26% |
| Microcrystalline cellulose | 90 | 19.34% |
| (Vivapur pH 102) | | |
| PEG 8000 | 18 | 3.85% |
| Povidone (PVP K-25) | 6 | 1.28% |
| Magnesium stearate | 2 | 0.43% |
| Tablet weight: | 468 | 100.0% |

### Preparation

Coenzyme Q-10, surfactant (PEG stearate) and oil phase (alpha-tocopherol acetate) were heated together between 50° C. and 55° C. and mixed until the coenzyme completely dissolved. This solution was diluted with ethyl alcohol and then mixed with colloidal silicon dioxide, dibasic calcium phosphate and part of microcrystalline cellulose as sorbents. The paste was carefully mixed to obtain homogenous dispersion. This is important to maintain a relatively uniform composition in the final tablet and also contributes to prolonged release and bioavailability. This dispersion was transferred to a planetary granulator and carefully mixed with gel-forming polymers Methocel K4M, Methocel E15 and part of lactose (hydration rate regulator). The mixture was granulated with separately prepared 5% binder solution of polyvinylpyrrolidone (Kollidon PVP K-25) in ethyl alcohol until a suitable granulate was obtained. This granulate was dried at 45° C. until the solvent evaporated. The dry granulate was passed through a (16 mesh) sieve, mixed with microcrystalline cellulose, lactose and sieved magnesium stearate (lubricant).

Tablets were prepared using conventional equipment (such as 16-station rotary tablet press). The tablets had a hardness of between 4 kg and 8 kg and friability of less than 1%.

Dissolution tests were carried according to USP requirements, using USP apparatus #2 at 37° C., with paddle rotation at 100 rpm. 900 ml of simulated gastric fluid (SGF) without enzymes or simulated intestinal fluid (SIF) served as the dissolution media.

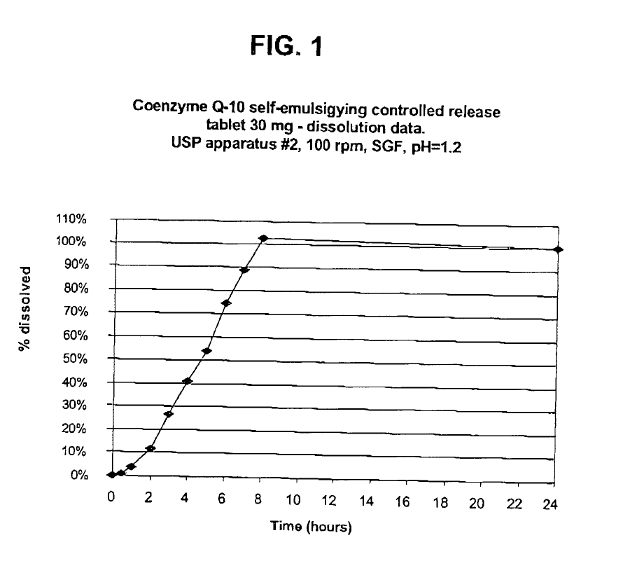

Dissolution was insensitive to media type. The tablet was almost completely dissolved between 6 and 8 hours. Upon dissolution, colloidal emulsion of the coenzyme Q-10 dissolved in the oil phase was formed and gradually released into dissolution media, forming a hazy bluish dispersion. The dissolution pattern is displayed in FIG. 1.

### EXAMPLE 2

Coenzyme Q-10 Self-Emulsifying Controlled Release Tablet (50 mg strength).

TABLE 2
Tablet Composition
Pharmaceutical Solid Self-Emulsifying Composition for
Sustained Delivery of Coenzyme Q-10 (50 mg tablet)

| | Per tablet, | |
|---|---|---|
| INGREDIENTS | mg | % |
| Coenzyme Q-10 | 50 | 7.06% |
| Tocopherol acetate | 50 | 7.06% |
| PEG-40 stearate | 80 | 11.30% |
| Dibasic calcium phosphate | 25 | 3.53% |
| Magnesium Aluminium Silicate | 75 | 10.59% |
| (Neusilin ®) | | |
| Microcrystalline cellulose | 125 | 17.65% |
| (Vivapur pH 102) | | |
| Methocel K4M | 35 | 4.94% |
| Methocel E-15 | 75 | 10.59% |
| Lactose (spray dried) | 150 | 21.18% |
| Povidone (PVP K-25) | 10 | 1.41% |
| PEG 8000 | 30 | 4.24% |
| Magnesium stearate | 3 | 0.42% |
| Tablet weight: | 708 | 100% |

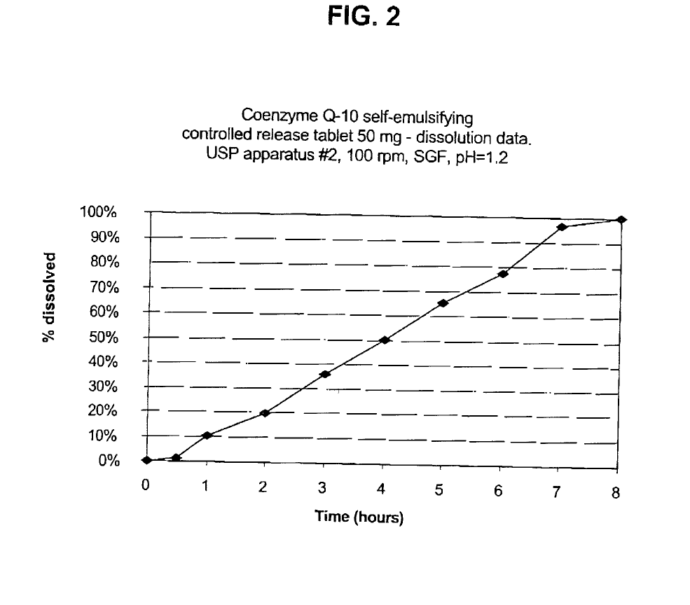

Preparation followed the protocol as described in Example 1. The tablet was found to be between 6 kg and 10 kg with a friability of less than 1%. The dissolution pattern is presented in FIG. 2.

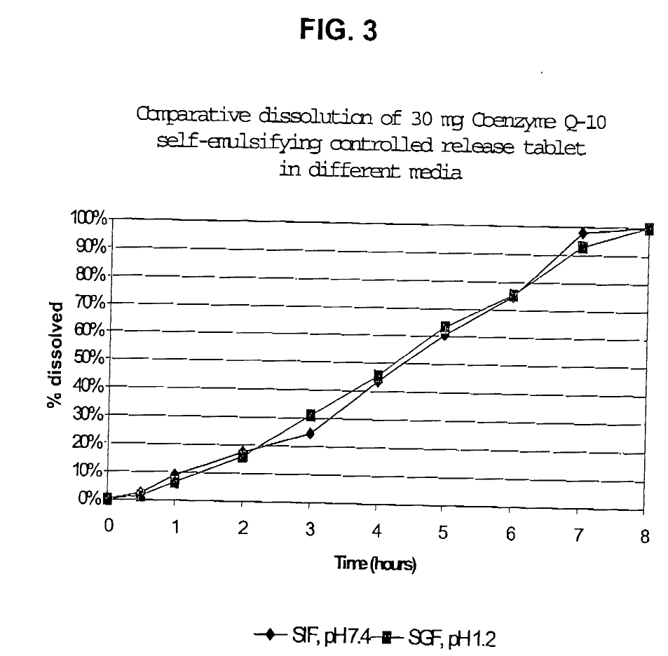

The drug release from self-emulsifying matrix can be absolutely independent to media type. FIG. 3 represents the dissolution pattern in acidic and basic conditions (simulated gastric and intestinal fluids without enzymes, according to USP 23).

### EXAMPLE 3

Alpha-lipoic acid in Self-Emulsifying Controlled Release Tablet (50 mg strength).

The slowly dissolving composition contained alpha-lipoic (octathioic) acid in amount of 50 mg per tablet. The oil phase comprised alpha-tocopherol acetate (vitamin E acetate). Another tocopherol derivative, tocopherol acid succinate PEG1000 ester (TPGS™) was used as the surfactant. The weight ratio between the lipoic acid and the oil phase used was 1:1. A 1:1 ratio was observed for the surfactant and oil phase.

The composition of the 50 mg extended release tablet is displayed in table 3.

TABLE 3
Solid Self-Emulsifying Pharmaceutical Composition for
Sustained Delivery of Alpha-Lipoic Acid

| | Per tablet, | |
|---|---|---|
| INGREDIENTS | mg | % |
| alpha-lipoic acid | 50 | 6.41% |
| alpha-Tocopherol acetate | 50 | 6.41% |
| TPGS (PEG1000-tocopherol | 50 | 10.68% |
| succinate) | | |
| Dibasic calcium phosphate | 15 | 3.21% |
| Colloidal silicon dioxide (Cab- | 45 | 9.62% |
| O-Sil) | | |
| Lactose (spray dried) | 110 | 23.50% |
| Methocel E-15 | 24 | 5.13% |
| Methocel K4M | 48 | 10.26% |
| Microcrystalline cellulose | 90 | 19.34% |
| (Vivapur pH 102) | | |
| PEG 8000 | 18 | 3.85% |
| Povidone (PVP K-25) | 6 | 1.28% |
| Magnesium stearate | 2 | 0.43% |
| Tablet weight: | | 100.0% |

### Preparation

Alpha-lipoic acid, alpha-tocopherol acetate and surfactant, alpha-tocopherol acid succinate-PEG1000 (TPGS™) were mixed together and stirred in dry ethanol until complete dissolution of the components was observed. The solution was then mixed with sorbents including colloidal silicon dioxide, dibasic calcium phosphate and part of microcrystalline cellulose. The paste formed was carefully mixed to achieve homogenous dispersion and transferred to a granulator and subsequently mixed with gel-forming polymers: Methocel K4M, Methocel E15 and part of lactose (hydration rate regulator). The formed blend was granulated with separately prepared 5% binder solution of polyvinylpyrrolidone (Kollidon PVP K-25) in ethyl alcohol until a proper granulate was obtained. This granulate was dried at 45° C. until the solvent was evaporated. The dry granulate was passed through a 16 mesh sieve, mixed with microcrystalline cellulose, lactose and sieved magnesium stearate (lubricant).

The tablets were prepared using the equipment as discussed in Example 1. The obtained tablet provided a hardness of between 5 kg and 8 kg with a friability of less than 1%.

Dissolution tests were carried according to USP requirements, using USP apparatus #2 at 37° C., with paddle rotation at 100 rpm. The tablet was completely dissolved in 6 hours. Upon dissolution a colloidal emulsion of oil droplets was formed and gradually released into the dissolution media, forming a hazy bluish dispersion. The active ingredient, alpha-lipoic acid, was distributed between the oil droplets and the water phase in accordance with the partition coefficient and pH of dissolution media.

The observed dissolution pattern was similar to that in the tablets of Examples 1 and 2.

### EXAMPLE 4

Indomethacin in self-emulsifying controlled release tablet (75 mg strength).

Indomethacin, a well known non-steroid antiinflammatory drug (NSAID), is very popular due to high potency of analgesic and antiflogistic action. A draw back of the compound is the side effect of a strong irritation of the gastric mucose. This is characterized of NSAIDS. By inclusion of the indomethacin (as other NSAID, e.g., diclofenac, piroxicam, naproxen, ketoprofen, etc.) into a self-emulsifying may decrease irritation due to contact of undissolved crystalline drug substance with sensitive stomach and intestine mucosal surfaces. The limited solubility of indomethacin in common oil phases required a suitable review of the composition of the oil phase components for better solubilization of the drug. As result of experimental probes, a mixture of MCT with polar oils, glycerol monolaurate and Labrafil™ 1944, was used. Tyloxapol™, a copolymer of alkylphenol and formaldehyde, was used as a pharmaceutical grade surfactant. A hydration rate controlling polymer, polyethylene oxide (Polyox™ WSR N-12K, Union Carbide) illustrated suitability of polyethylene oxide homopolymer for self-emulsifying controlled release matrices.

Compositional details of the 75 mg indomethacin self-emulsifying extended release tablet are displayed in table 4.

TABLE 4
Solid Self-Emulsifying Pharmaceutical Composition for
Sustained Delivery of Indomethacin (75 mg)

| | Per tablet, | |
|---|---|---|
| INGREDIENTS | mg | % |
| Indomethacin | 75 | 8.85% |
| Miglyol 812 (MCT oil) | 140 | 16.53% |
| Glycerol monolaurate (GML) | 180 | 21.25% |
| Labrafil ™ 1944 | 80 | 9.45% |
| Tyloxapol ™ | 40 | 4.72% |
| Sodium Aluminium Silicate | 60 | 7.08% |
| Colloidal silicon dioxide | 40 | 4.72% |
| (Aerosil ™ 300) | | |
| Lactose (spray dried) | 60 | 7.08% |
| Polyox ® WSRN 12K | 100 | 11.81% |
| (Polyethylene oxide 2 mln) | | |
| Microcrystalline cellulose | 60 | 7.08% |
| (Avicel pH 101) | | |
| Povidone (PVP K-90) | 10 | 1.18% |
| Magnesium stearate | 2 | 0.24% |
| Tablet weight: | 847 | 100.00% |

Indomethacin, MCT oil, Labrafil 1944 and glycerol monolaurate (GML) and surfactant Tyloxapol™ were mixed together and heated to between 55° C. and 60° C. until a clear solution was obtained. The solution was then mixed with the sorbents colloidal silicon dioxide, sodium aluminium silicate and part of microcrystalline cellulose. The formed paste was carefully mixed to homogeneity. This dispersion was granulated and mixed with the gel-forming polymer Polyox WSR N-12K and part of lactose (hydration rate regulator). The formed blend was granulated with a separately prepared 5% binder solution of polyvinylpyrrolidone (Kollidon PVP K-90) in ethyl alcohol until a proper granulate was obtained. This granulate was dried at 45° C. until the solvent was totally evaporated.

The granulate was sieved (16 mesh), mixed with rest part of microcrystalline cellulose, lactose and sieved magnesium stearate (lubricant). Capsule shaped tablets were prepared to yield tablets having a hardness between 3.5 kg and 4.5 kg.

The dissolution tests were carried according to USP requirements, using USP apparatus #2 at 37° C., with paddle rotation at 100 rpm. Complete dissolution of the tablet was achieved in 6 hours. Upon dissolution a colloidal emulsion of the oil droplets was formed and gradually released into dissolution media, forming hazy bluish dispersion. The active component, indomethacin, was distributed between the oil droplets and water phase in accordance with the partition coefficient and pH of the dissolution media.

A controlled release self-emulsifying tablet comprising 25 mg of indomethacin was prepared by similar manner as Example 4, but with another composition. (See Table 5).

TABLE 5
Solid Self-Emulsifying Pharmaceutical Composition for
Sustained Delivery of Indomethacin (25 mg)

| | Per tablet, | |
|---|---|---|
| INGREDIENTS | mg | % |
| Indomethacin | 25 | 3.39% |
| Tocopherol acetate | 80 | 10.84% |
| Imwitor ™ 308 (Glycerol | 80 | 10.84% |
| monocaprylate) | | |
| Mirj ® 52 | 80 | 10.84% |
| Colloidal silicon dioxide | 100 | 13.55% |
| (Cab-O-Sil) | | |
| Dibasic calcium phosphate | 80 | 10.84% |
| Hydroxypropylmethylcellulose | 80 | 10.84% |
| (Methocel E-50) | | |
| Lactose (spray dried) | 120 | 16.26% |
| Microcrystalline cellulose | 60 | 8.13% |
| (Vivapur pH 102) | | |
| Povidone (PVP K-25) | 10 | 1.36% |
| PEG 3350 | 20 | 2.71% |
| Magnesium stearate | 3 | 0.41% |
| Tablet weight: | 738 | 100.00% |

This tablet has satisfactory physical properties (hardness, friability, tabletting behavior) and dissolution profile.

The developed delivery system can be successfully applied for controlled release of natural active substances, both plant and animal origin. The best results were observed with extracts.

### EXAMPLE 5

Self-emulsifying controlled release tablet with 50 mg of Red Reishi Mushrooms extract.

The Red Reishi Mushroom demonstrates high activity as immunomodulator and use as a nutritional additive. Recently, extract of the mushrooms was presented to replace multiple bulky doses (600 mg capsules 3-4 times a day) for 20-50 mg of dry material concentrate of active ingredients. The main active components in the extract are different triterpenoids, aromatic compounds and polysaccharides.

The tablet allowed a significantly improved drug release pattern and consumer convenience. It was found that one tablet a day provided constant and smooth delivery of the active ingredients. In the process of dissolution, oil droplets loaded with triterpenoids and surrounded by polysaccharides were formed and found to efficiently penetrate the gastrointestine to provide a supply of the biologically active ingredients.

TABLE 6
Composition of Self-Emulsifying Controlled Release Tablet
with 50 mg of Red Reishi Mushrooms Extract

| COMPONENT | Per tablet, mg | % |
|---|---|---|
| Red mushrooms “REISHI” extract | 50 | 9.40% |
| Alpha-Tocopherol acetate | 25 | 4.70% |
| TPGS ™ (PEG1000 tocopherol | 25 | 4.70% |
| succinate) | | |
| Colloidal silicon dioxide | 50 | 9.40% |
| (Syloid ® 244, GRACE) | | |
| Dibasic calcium phosphate | 100 | 18.80% |
| Methocel E-15 | 40 | 7.52% |
| Methocel K4M | 60 | 11.28% |
| PVP K-25 | 10 | 1.88% |
| PEG-8000 | 20 | 3.76% |
| Lactose spray dried | 100 | 18.80% |
| Microcrystalline cellulose | 50 | 9.40% |
| Magnesium stearate | 2 | 0.38% |
| Tablet weight | 532 | 100% |

Granulation was prepared as described in accordance with Example 2, but the granulate was dried at between 32° C. and 35° C.

The extract of Red Reishi Mushrooms (Garuda Inc., USA), alpha-tocopherol acetate and surfactant, alpha-tocopherol acid succinate-PEG1000 (TPGS™, Eastman) were mixed together and stirred in dry ethanol at 35° C. until a homogenous suspension was obtained. The suspension was mixed with sorbents as in the previous examples. The formed paste was carefully mixed, transferred to the granulator and mixed with Methocel K4M, Methocel ElS and PVP. The formed blend was then granulated with ethyl alcohol until a proper granulate was obtained. The granulate was dried at temperature no more than 35° C. (to prevent evaporation of volatile aromatic compounds of extract) until the solvent was totally evaporated.

The dried granulate was sieved and mixed with microcrystalline cellulose, inter alia as discussed previously. The tablets were found to have a hardness of between 8 kg and 10 kg and a friability of less than 1%.

The tablet determined in accordance with USP 23 (37° C., 100 rpm, 900 ml water) dissolved in about 6 hours in apparatus 2 (more than 80% dissolved).

### EXAMPLE 6

Multivitamin composition in self-emulsifying controlled release tablet.

The formulation included water soluble and a lipid soluble vitamin components and was prepared consistent with the method described in Example 3. The composition is presented in table 7.

TABLE 7
Self-emulsifying controlled release tablet formulation for
water soluble and lipid soluble vitamins.

| INGREDIENTS | Per tablet, mg | % |
|---|---|---|
| Ascorbyl palmitate (Vitamin C) | 50 | 5.88% |
| Alpha-Tocopherol acetate (Vitamin E) | 160 | 18.82% |
| Retinol acetate (Vitamin A) | 4.5 | 0.53% |
| 10,000 I.U. | | |
| TPGS (Vitamin E) | 51.7 | 6.08% |
| Tocopherol acid succinate | 25 | 2.94% |
| (Vitamin E) | | |
| Calcium ascorbate (Vitamin C) | 165 | 19.41% |
| Magnesium Aluminium Silicate | 60 | 7.06% |
| (Neusilin UHL-2) | | |
| Dibasic calcium phosphate | 80 | 9.41% |
| Microcrystalline cellulose | 40 | 4.71% |
| Methocel E-15 | 60 | 7.06% |
| Methocel K4M | 20 | 2.35% |
| PVP K-25 | 10 | 1.18% |
| PEG-8000 | 20 | 2.35% |
| Lactose spray dried | 60 | 7.06% |
| Microcrystalline cellulose | 40 | 4.71% |
| Magnesium stearate | 3.8 | 0.45% |
| Tablet weight: | 850 | 100.00% |

The main advantage of sustained release delivery of self-emulsifying compositions is realized by the highly increased bioavailability of the included active components. This is of great importance for poorly soluble compounds and controlled delivery of such compounds can significantly decrease potentially dangerous drug dumping and provide constant and uniform delivery profiles.

Entrapping the drug into the small (usually less than 5-10 micron diameter) oil droplets leads to significantly decreased local irritation (it is extremely important for such drugs as NSAID) and visibly increases penetration efficacy through the gastro-intestinal mucosal membranes. Absence of undissolved NSAID crystals adhered on the stomach wall eliminates possible bleeding due to drug erosive action.

In view of the fact that the pattern of the size distribution for these oil droplets is similar to chylomicrons it is reasonable to suppose corresponding behavior in the gastro-intestinal system and expect improved absorption of the drug, included in the oil phase by an analogous mechanism.

The described pharmaceutical composition has sufficient loading of the poor water-soluble drug, and provides prolonged release of the included drug. The drug loaded oil-in-water emulsion is gradually released from the composition.

Different types of active compounds were successfully incorporated into the composition, this demonstrating that the composition has wide suitability and potential for different types of biologically active materials.

Conveniently, sustained release of the active material permits a change from multiple dosing (2-6 tablets a day) to a single dose delivery per day. This feature decreases the chances for missing doses or significant variations of the drug in the blood.

### Pharmacokinetics of Coenzyme Q-10 in Self-Emulsifying Tablet

The CoQ10 pharmacokinetics for self-emulsifying tablet as set forth in Example 2 was investigated relative to the only available 50 mg CoQ10 tablet (Enzymatic Therapy®, CoQ10 50 mg, lot L9300). This tablet contains micronized CoQ10.

Conducted with twenty healthy male volunteers (aged 19˜23 years) participated.

Each subject of one group received multiple oral doses of coenzyme Q₁₀ as sustained release tablets for fifteen days and each day took one time with 50 mg. The subjects of the other group did the same, but with regular tablets.

The blood samples were taken prior to the oral administration and at specified times. After blood plasma was precipitated by methanol for protein removal it was extracted with hexane. Aqueous and organic solvents were separated by low speed centrifugation and the organic phase was collected, dried under a nitrogen gas stream and dissolved into 100 μl of ethanol. The solution was injected into HPLC-UV system with a 10 μm, 250 μ×4.6 mm reverse phase column and heated to 30° C. The mobile phase was constituted by methanol-ethanol 9:1 v/v with a flow rate of 1.5 ml·min−1 and UV detection at 275 nm. Coenzyme Q₉ was used as an internal standard material for analysis.

### Results

Total coenzyme Q10 concentrations in plasma following oral administration of self-emulsified tablets were higher (p<0.05), compared to those in plasma following oral administration of regular tablets. According to obtained pharmacokinetic data, blood concentration of CoQ10 at day 14 increased from initial level ˜50% for commercial immediate release tablet and ˜80% for self-emulsifying tablet. AUC values are 146% and 188%, respectively (100%—initial CoQ10 level, 0.81 and 0.96 mcg/ml, resp.).

| | Self-emulsifying | | “Enzymatic Therapy” | |
|---|---|---|---|---|
| | tablet | | Lot L9300 | |
| | 50 mg Coenzyme | | micronized CoQ10 50 | |
| | Q-10 | | mg | |
| Cmax | 1.85 | mcg/ml (day 14) | 1.37 | mcg/ml (at day 7) |
| Relative AUC | 361 | mcg*hr/ml | 193 | mcg*hr/ml |

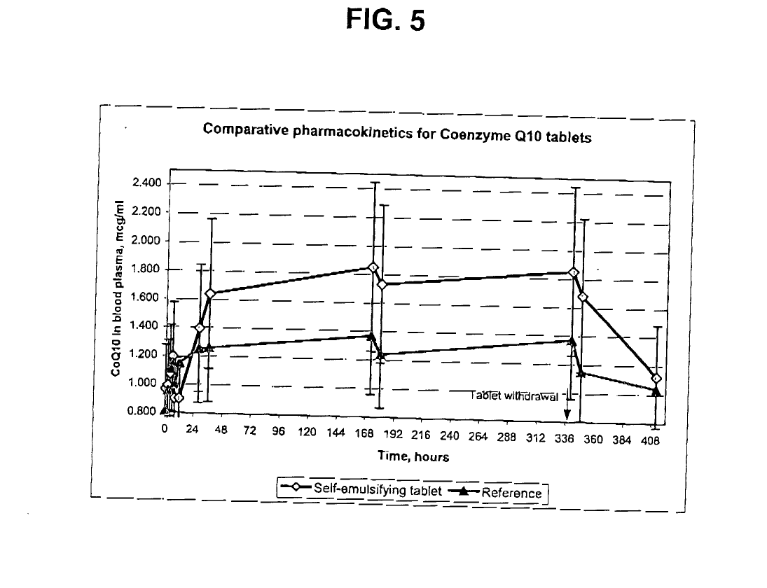

FIG. 5 represents change in CoQ10 concentration in blood plasma.

Although embodiment of the invention have been described above, it is not limited thereto and it will be apparent to those skilled in the art that numerous modifications form part of the present invention insofar as they do not depart from the spirit, nature and scope of the claimed and described invention.

I claim:

1. A solid oral dosage form for improved bioavailability of poorly water soluble hydrophobic compounds, providing “in situ” formation and release of oil-in-water emulsion on contact with water containing media.

2. Solid dosage form as set forth in claim 1, prepared as compressed tablet or hard gelatin capsule.

3. Solid dosage form as set forth in claim 2, wherein biologically active compounds releases being dissolved or dispersed in oil droplets of “in situ” forming oil-in-water emulsion.

4. Solid dosage form as set forth in claim 3, wherein named emulsion comprises of oil droplets with particle size from 0.01 to 100 micron, preferably from 0.1 to 10 micron.

5. Solid dosage form as set forth in claim 2, comprises of:
(i) at least one biologically active material;
(ii) named material is dissolved, dispersed, or uniformly suspended in physiologically acceptable hydrophobic phase;
(iii) at least one surfactant providing emulsification of the hydrophobic phase after contact with water media; and
(iv) at least one physiologically acceptable sorbent to incorporate hydrophobic phase.

6. A composition for manufacturing of solid dosage form as set forth in claim 5, where named hydrophobic phase is liquid or semisolid at body temperature

7. A composition for manufacturing of solid dosage form as set forth in claim 6, where named hydrophobic phase absorbed on the named sorbent.

8. A composition for manufacturing of solid dosage form as set forth in claim 8, where named hydrophobic phase is not squeezed from the sorbent during tablet compression step.

9. Solid dosage form as set forth in claim 8, wherein ratio between sorbent and hydrophobic phase is in range from 1:to 10 to 10:1, preferably in range 1:3 to 3:1.

10. A composition of claim 5, where named hydrophobic phase comprises of compound, selected from pharmaceutical or food grade oils and fats (soya oil, olive oil, kernel oil, cocoa butter, jojoba oil, fish oil, etc.).

11. A composition of claim 5, where named hydrophobic phase comprises of at least one compound, selected from group of pharmaceutically acceptable glycerides and glycerin saturated and unsaturated fatty acid (C2-C22) esters (Medium Chain Triglycerides, tricaprin, trimyristin, triolein), mono- and diglycerides, their mixtures and derivatives (Capmul™, Miglyol™, Myvacet™, Witepsol™, Imwitor™, Dynasan™, Crodamol™).

12. A composition of claim 5, where named hydrophobic phase comprises of at least one compound, selected from group of fatty and aliphatic acid and fatty acids esters (oleic and linoleic acid, ethyl oleate, ethyl linoleate, isopropylmyristate, propyleneglycol C2-C12 esters, ethylpalmitate, isopropylpalmitate, isostearic esters, diethyladipate, diethylsebacate triethylcitrate, ethyltributylcitrate, dioctylphtalate).

13. A composition of claim 5, where named hydrophobic phase comprises of lipidic pharmaceutically acceptable compounds (alpha-, beta and gamma-tocopherols, tocopherol acetate, tocopherol nicotinate, retinol acetate, retinol palmitate, cholesteryl esters, stearyl alcohol, sucrose acetate isobutyrate).

14. A composition of claim 5, where named hydrophobic phase comprises of phospholipid compound (soy and egg lecithin and analogs) or a mixture of phospholipids selected from the group consisting of phosphatidylcholine, phosphatidylethanolamine, phosphatidylserine, phosphatidylinositol, phosphatidylglycerol, phosphatidic acid, sphingomyelin.

15. A composition of claim 5 comprises at least one surfactant, selected from group of polyoxyethylated compounds such as polyoxyethylated fatty acids (PEG-stearates, PEG-laurate), PEG-ethers, sorbitan derivatives (Tween™), aromatic polyoxyethylated compounds (Triton X-100, Tyloxapol), PEG-glycerides (PECEOL), PEG-PPG copolymers (Pluronic®, Poloxamers), Polyglycerines, PEG-tocopherols, propylene glycol derivatives.

16. A composition of claim 5 comprises at least one surfactant, selected from group of sugar, polysaccharide or polyol alkyl and acyl derivatives (octylsucrose, octylglucose, octylmannoside, sucrose stearate, and lauroyldextran).

17. A composition of claim 5 comprises at least one surfactant, selected from group of anionic compounds such as soaps (sodium stearate, sodium caproate, sodium stearyl fumarate) or alkylsulfonates (sodium dodecylsulfate) composition of claim 5 comprises at least one surfactant, selected from group of amphoteric surfactants.

18. A composition of claim 5 where named hydrophobic phase absorbed on the particles of at least one acceptable sorbent, selected from the group of silicon dioxide (Aerosil™, Cab-O-Sil™, Syloid™, Sipernat™) or inorganic salts: calcium, magnesium and aluminium silicates (Neusilin™), di-and tribasic calcium phosphates, calcium sulphate.

19. A composition of claim 5, where excipient selected from group of water insoluble polymers, such as microcrystalline cellulose, amorphous cellulose, milled cellulose, starch, dextrin, crosslinked polyvinylpyrrolidon.

20. A composition of claim 5, where excipient selected from group of water soluble sugars, polysaccharides and polyols, such as lactose, sucrose, fructose, mannitol, xylitol, sorbitol.

21. A composition of claim 5, where excipient selected from group of water soluble polymers such as hydroxypropylmethylcellulose, methylcellulose, hydroxyethylcellulose, hydroxypropylcellulose, caboxymethylcellulose, polyacrylic acid, alginic acid, hyaluronic acid, pblygalacturonic acid, polymannuronic acid, xantan gum, locust beam gum, carrageenan, caraya gum, acacia gum, chitosan, polyethylene oxide, polyvinylpyrrolidone and copolymers, polyvinyl alcohol.

22. A process for preparation of composition of claim 1, includes distribution of the active material and surfactant in hydrophobic base, blending of the formed mixture with sorbent(s),following addition of the other excipients, granulation and preparation of the tablet using tablet press machine.

23. A process of claim 22, where active material is dissolved or dispersed in melted mixture of hydrophobic phase and surfactants and then mixed with a sorbent.

24. A process of claim 22, where granulation is prepared by compacting of sorbent with active components, hydrophobic phase with surfactant(s) and other excipients using compacting or slugging equipment.

25. A process of claim 22, where active material is granulated with other components using volatile solvent.

26. A process of claim 25, where volatile solvent is selected from group of methyl alcohol, ethyl alcohol, propyl alcohol, isopropyl alcohol, butyl alcohol, isobutyl alcohol, tert-butyl alcohol, acetone, methylethylketone, ethyl acetate, amylacetate, isopropyl acetate, toluene, xylol, metylene chloride, trichlormethane, tetrachlormethane, methane, dichloroethane, purified water and water-alcohol mixtures.
